(19) United States
(12) Patent Application Publication
(10) Pub. No.: US 2024/0071596 A1
(43) Pub. Date: Feb. 29, 2024

(54) RECOVERY PROGRESSION AND MOVEMENT TIMELINES FROM NETWORK-CONNECTED SENSOR DEVICES

(71) Applicant: AT&T Intellectual Property I, L.P., Atlanta, GA (US)

(72) Inventors: Nigel Bradley, Canton, GA (US); Rashmi Palamadai, Naperville, IL (US)

(21) Appl. No.: 17/899,968

(22) Filed: Aug. 31, 2022

(51) Int. Cl. G16H 20/30; G06N 20/00

(52) U.S. Cl. CPC G16H 20/30; G06N 20/00

(57) ABSTRACT

A processing system may obtain first inputs from at least one sensor device, where the first inputs comprise at least a first visual input, apply the first inputs to a monitoring model for monitoring a particular type of movement activity of a user, the monitoring model configured to detect at least one trigger condition, and obtain an output of the monitoring model in accordance with the first inputs, the output indicates the at least one trigger condition is detected. The processing system may next obtain second inputs from the at least one sensor device, the second inputs comprising at least a second visual input, apply the second inputs to a recovery model associated with the at least one trigger condition, obtain an output of the recovery model in accordance with the second inputs, the output indicating an advancement along a therapy progression, and present a notification of the advancement.

The present disclosure relates generally to network-connected sensor devices, and more particularly to methods, computer-readable media, and apparatuses for presenting a notification of an advancement along a therapy progression based on an output of a recovery model in accordance with a plurality of inputs comprising at least a visual input from at least one sensor device.

### BRIEF DESCRIPTION OF THE DRAWINGS

The teachings of the present disclosure can be readily understood by considering the following detailed description in conjunction with the accompanying drawings, in which:

FIG. 1 illustrates an example system related to the present disclosure;

FIG. 2 illustrates an example process in accordance with the present disclosure;

FIG. 3 illustrates a flowchart of an example method for presenting a notification of an advancement along a therapy progression based on an output of a recovery model in accordance with a plurality of inputs comprising at least a visual input from at least one sensor device; and

FIG. 4 illustrates a high level block diagram of a computing device specifically programmed to perform the steps, functions, blocks and/or operations described herein.

To facilitate understanding, identical reference numerals have been used, where possible, to designate identical elements that are common to the figures.

### DETAILED DESCRIPTION

Methods, computer-readable media, and apparatuses for presenting a notification of an advancement along a therapy progression based on an output of a recovery model in accordance with a plurality of inputs comprising at least a visual input from at least one sensor device are described. For example, a processing system including at least one processor may obtain a first plurality of inputs from at least one sensor device associated with a user, where the first plurality of inputs comprises at least a first visual input. The processing system may then apply the first plurality of inputs to a monitoring model for monitoring a particular type of movement activity of the user, where the monitoring model is configured to detect at least one trigger condition in accordance with the first plurality of inputs, and obtaining an output of the monitoring model in accordance with the first plurality of inputs, wherein the output indicates the at least one trigger condition is detected. The processing system may next obtain a second plurality of inputs from the at least one sensor device, wherein the second plurality of inputs comprises at least a second visual input, apply the second plurality of inputs to a recovery model associated with the at least one trigger condition, and obtain an output of the recovery model in accordance with the second plurality of inputs, where the output indicates an advancement along a therapy progression. The processing system may then present a notification of the advancement along the therapy progression.

In particular, examples of the present disclosure may trigger input from ecosystem devices based on user behavior, and may capture and store visual data, detect anomalies in the visual data, and share snapshots with the user and/or healthcare professional to confirm an anomaly that may trigger a recovery phase. For instance, in one example, the present disclosure may detect a type of injury from analysis of visual data (and in one example, additional audio data and/or biometric data), and may present at least one option and/or create a feedback loop for the benefit of the user, and with added convenience to healthcare professionals. In one example, the present disclosure presents a candid snapshot and progressive view of a user's health, in particular user mobility, which may be on a pre-determined schedule, and which may account for a user profile, genetic predisposition, lifestyle choices, and other factors. In one example, the present disclosure may integrate with or may comprise part of a health assistant application, and may be in communication with ecosystem devices to pull, probe, request and share information.

Examples of the present disclosure may detect that a user has had an accident, is recovering from a medical procedure, is starting a new exercise routine or new overall routine (e.g., walking instead of driving to work), is starting a new job with physical impact, and so forth. In one example, the present disclosure may provide a timeline of a user's movement history to enable the user to understand past and current state of health, and to project where the user is heading for the user's health and well-being. The timeline may present a “story” of the user's personal health journey, which may be used for either self-monitoring, or to tie-in with a healthcare professional's evaluation of the user's progress in a recovery (or possible regression or further injury). The timeline may present a cohesive, easy to understand movement history, with visual (or audiovisual) clips documenting the user's movement to allow the user and/or healthcare professionals to understand potential impacts due to exertions/activities, or overall status of health.

In one example, the present disclosure may include an initial baselining of the user's movement(s). For instance, the user may begin with an initial complaint, an injury, or a post-operation baseline. Alternatively, the user may initiate ongoing monitoring from a healthy state. In one example, the baselining may include identifying available sensor devices such as cameras, microphones, wearable biometric devices, or the like, the locations of such devices (e.g., if fixed locations), etc., from which the present disclosure may access user information. In one example, the present disclosure may integrate with ecosystem devices to pull, probe, request and share information. In one example, the present disclosure may obtain data from ecosystem devices via a virtual health assistant that may already have access to such devices and the data thereof. Alternatively, or in addition, examples of the present disclosure may be integrated with or comprise part of a virtual health assistant. In one example, the present disclosure may probe for missing information and incorporate user input to create a baseline picture.

In a monitoring phase, the system may notice a potential injury, a change in behavior, or a change in movement (e.g., change in gait, or the like), and may probe for more information. In one example, for an apparent injury (e.g., detected in visual data or specifically indicated by the user), the present disclosure may confirm a recovery plan with the user and/or health professional. In one example, a recommended recovery plan may be based on medical records, user profile, age, overall health, etc. For a non-apparent injury or other anomalies (e.g., a measurable change in gait, range of motion, etc. over a period of time, such as more than one week, more than two weeks, etc.), the present disclosure may also query for more information and may confirm an anomaly based on user and/or health professional feedback, e.g., “It appears range of motion for overhead lift is restricted, are you injured or do you have pain?,” or the like. In one example, in the monitoring phase, the present disclosure may also identify deviations in correct performance of motion activities that do not necessarily indicate a limitation of the user's mobility or overall health, but which may be a risk factor for injury or deterioration. In such case, the user may be warned that if the movement continues, the user may incur higher risk of injury/health impact. In one example, the present disclosure may alternatively or additionally access historical medical records for the user and/or family members to show trend lines towards progressive conditions based on a personalized profile.

In one example, during the monitoring phase, a timeline of the user's movement history may be sent to a health professional to assist in identifying pain, or changes in a pain level, identifying changes in movement, and so forth. For instance, as noted above, one example of detecting an apparent injury is via explicit input by a health professional. Thus, interventions may be initiated prior to a user actually seeking medical attention. In one example, the timeline may include descriptions of patterns to point out to the user, and which a health professional can address upon review (e.g., immediately) or at a next visit by the user. For instance, the timeline may include a marker that indicates “overhead reach limited to 30%” and the date and/or time at which this limitation of the user's motion was detected. In addition, the system may log and record user pain and complaints over time, which may also be included in the timeline. In one example, the present disclosure provides a feedback loop, e.g., via a user dialog, that learns from user behavior and queries for more information to determine possible causes. In one example, this may help distinguish between psychological versus physical problems. For instance, the present disclosure may query the user about a detected deterioration in range of motion. However, the user may indicate that there is no pain or injury. Rather the user may just be tired and may choose not to exert too aggressively, e.g., intentionally moving more gingerly instead.

It should be noted that in one example, the user may approve specific linking or sharing of details, and may include use-defined selections for time-based presentation of mobility/pain reports, e.g., timeline(s). For instance, a user may share an entire timeline (e.g., all available/retained data, or all available/retained data relating to a particular aspect of the user's mobility or overall health (e.g., all related to upper body movement)) with a health professional, but may share only data from one week prior to an injury until two months after the injury with an insurance company.

After detection of a triggering condition, e.g., an injury, a regression/deterioration of mobility, or the like, the present disclosure may initiate a recovery phase. For example, the present disclosure may recommend or a health professional may direct a specific therapy progression. In one example, the user and/or health professional may confirm the selected therapy progression/recovery path, e.g., if automatically recommended by the system. In one example, the therapy progression may include a schedule of expected performance of one or more movement activities over time, e.g., over the course of days, weeks, or months. In one example, the user's mobility may continue to be measured via ecosystem devices for advancement along the therapy progression, e.g., 30 percent of maximum overhead extension after one week, 75 percent of maximum overhead extension after one month, full recovery after 3 months, etc. In one example, visual or other data gathered from ecosystem devices may continue to be recorded and may be added to the timeline for presentation to the user and/or health professional.

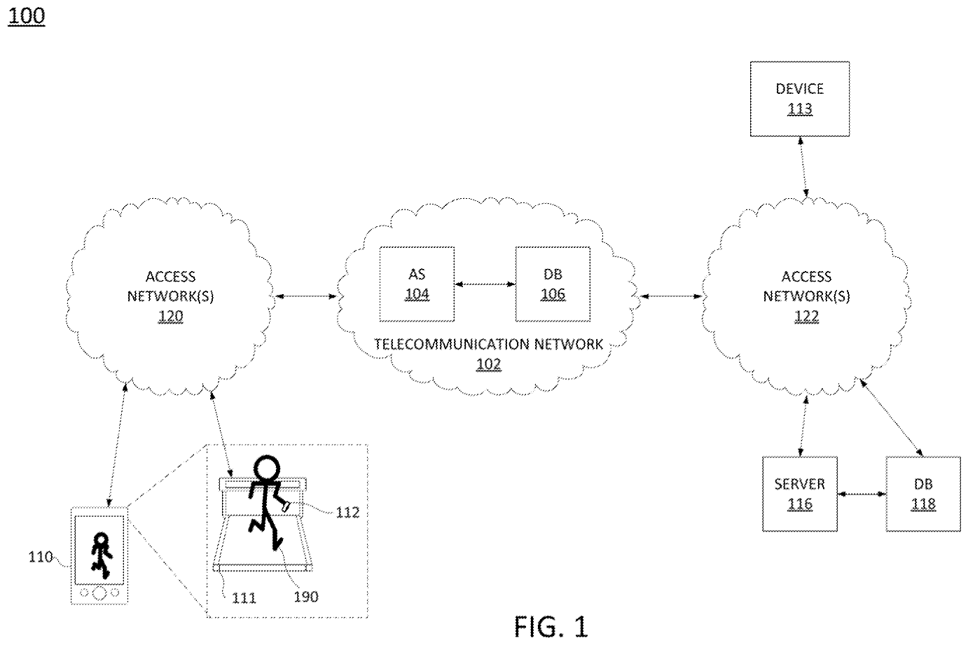

Thus, examples of the present disclosure help a user to understand the user's mobility and overall health and of well-being, e.g., past and present as well as future/expected. Advantageously, the present disclosure provides a timeline or story for a user's personal self-monitoring, and may tie-in with a health professional's evaluation of user progress in a therapy progression (or possible regression or further injury). Through recovery monitoring via visual and/or other data captured from ecosystem devices, the present disclosure may substantiate whether the user's healing is on target or may be delayed. In addition, a health professional can be alerted and may intervene in a timely manner, instead of the user waiting for an appointment/visit. In particular, user data, e.g., in the form of a timeline as described herein, can be sent to the professional at regular intervals, upon request, and/or pre-scheduled to align with an office visit. In addition, examples of the present disclosure provide a candid picture of mobility and overall health. For example, a timeline including only a few snapshots may unequivocally indicate that the user is not engaging in substantial activity. This puts the knowledge in the user's hands so that the user may understand the impact of the user's actions (or lack of action). In addition, the timeline provides a clear and concise picture of mobility (and/or overall health) for the user or health professional such that accurate analysis/decisions can be made. Notably, users, healthcare providers, insurance entities, and others can track and correlate mobility and overall health progression/regression through detailed timeline records and system generated presentations. These and other aspects of the present disclosure are described in greater detail below in connection with the examples of FIGS. 1-4.

To further aid in understanding the present disclosure, FIG. 1 illustrates an example system 100 in which examples of the present disclosure for presenting a notification of an advancement along a therapy progression based on an output of a recovery model in accordance with a plurality of inputs comprising at least a visual input from at least one sensor device may operate. The system 100 may include any one or more types of communication networks, such as a traditional circuit switched network (e.g., a public switched telephone network (PSTN)) or a packet network such as an Internet Protocol (IP) network (e.g., an IP Multimedia Subsystem (IMS) network), an asynchronous transfer mode (ATM) network, a wireless network, a cellular network (e.g., 2G, 3G, 4G, 5G and the like), a long term evolution (LTE) network, and the like, related to the current disclosure. It should be noted that an IP network is broadly defined as a network that uses Internet Protocol to exchange data packets. Additional example IP networks include Voice over IP (VoIP) networks, Service over IP (SoIP) networks, and the like.

In one example, the system 100 may comprise a telecommunication network 102. The telecommunication network 102 may be in communication with one or more access networks 120 and 122, and the Internet (not shown). In one example, telecommunication network 102 may combine core network components of a cellular network with components of a triple play service network; where triple-play services include telephone services, Internet services and television services to subscribers. For example, telecommunication network 102 may functionally comprise a fixed mobile convergence (FMC) network, e.g., an IP Multimedia Subsystem (IMS) network. In addition, telecommunication network 102 may functionally comprise a telephony network, e.g., an Internet Protocol/Multi-Protocol Label Switching (IP/MPLS) backbone network utilizing Session Initiation Protocol (SIP) for circuit-switched and Voice over Internet Protocol (VoIP) telephony services. Telecommunication network 102 may further comprise a broadcast television network, e.g., a traditional cable provider network or an Internet Protocol Television (IPTV) network, as well as an Internet Service Provider (ISP) network. In one example, telecommunication network 102 may include a plurality of television (TV) servers (e.g., a broadcast server, a cable head-end), a plurality of content servers, an advertising server (AS), an interactive TV/video on demand (VoD) server, and so forth. For ease of illustration, various additional elements of network 102 are omitted from FIG. 1.

In one example, the access networks 120 and 122 may comprise Digital Subscriber Line (DSL) networks, public switched telephone network (PSTN) access networks, broadband cable access networks, Local Area Networks (LANs), wireless access networks (e.g., an Institute for Electrical and Electronics Engineers (IEEE) 802.11/Wi-Fi network and the like), cellular access networks, 3^{rd} party networks, and the like. For example, the operator of telecommunication network 102 may provide a cable television service, an IPTV service, or any other types of telecommunication service to subscribers via access networks 120 and 122. In one example, the access networks 120 and 122 may comprise different types of access networks, may comprise the same type of access network, or some access networks may be the same type of access network and other may be different types of access networks. In one embodiment, the telecommunication network 102 may be operated by a telecommunication network service provider. The telecommunication network 102 and the access networks 120 and 122 may be operated by different service providers, the same service provider or a combination thereof, or may be operated by entities having core businesses that are not related to telecommunications services, e.g., corporate, governmental or educational institution LANs, and the like.

In one example, the access networks 120 may be in communication with one or more devices e.g., devices 110-112. Similarly, access networks 122 may be in communication with one or more devices, e.g., device 113, server 116, database (DB 118), and so forth. Access networks 120 and 122 may transmit and receive communications between devices 110-113, between devices 110-113, and server 116 and/or database (DB) 118, application server 104 and/or database (DB) 106, other components of telecommunication network 102, devices reachable via the internet in general, and so forth. In one example, each of the devices 110 and 113 may comprise any single device or combination of devices that may comprise a user endpoint device. For example, the devices 110 and 113 may each comprise a mobile device, a cellular smart phone, a laptop, a tablet computer, a desktop computer, an application server, a bank or cluster of such devices, and the like. For example, device 110 of user 190 may comprise a tablet computer, cellular smartphone and/or non-cellular wireless device, or the like with at least a camera and a display.

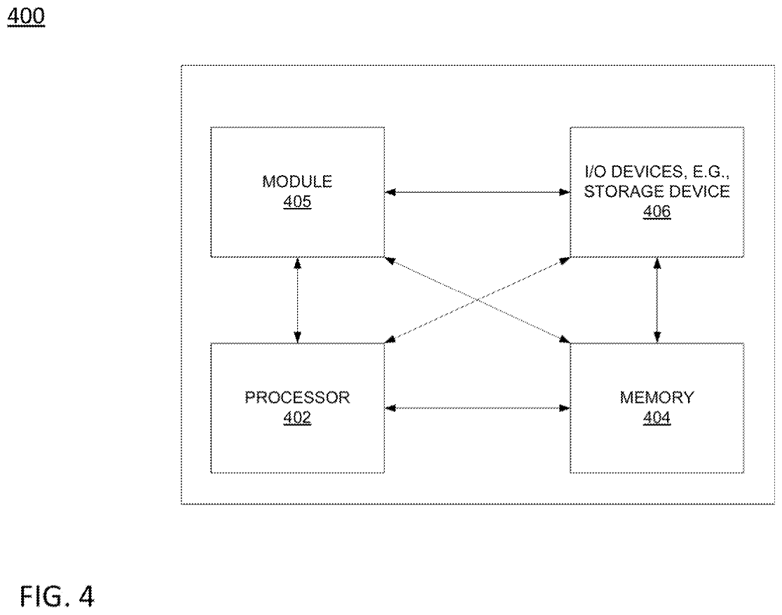

In one example, device 110 may comprise programs, logic or instructions for performing functions in connection with examples of the present disclosure for presenting a notification of an advancement along a therapy progression based on an output of a recovery model in accordance with a plurality of inputs comprising at least a visual input from at least one sensor device. For example, device 110 may comprise a computing system or device, such as computing system 400 depicted in FIG. 4, and may be configured to provide one or more operations or functions in connection with examples of the present disclosure for presenting a notification of an advancement along a therapy progression based on an output of a recovery model in accordance with a plurality of inputs comprising at least a visual input from at least one sensor device, as described herein. For instance, device 110 may comprise a mobility tracking and recovery/therapy progression application (app), e.g., associated with a network-based service, or system for mobility tracking and recovery/therapy progression.

Device 111 may comprise an exercise machine or a physical therapy machine (e.g., a treadmill) including network-based and/or peer-to-peer communication capabilities and may report speed, distance, stride length, or other factors (e.g., biometric data) to one or more other devices or systems, such as device 110, server 116, and so forth. Device 112 may comprise a wearable biometric monitoring device that may record measurements of the biometric data of user 190 while running at the various speeds or performing other movement activities (e.g., heart rate, breathing rate, skin temperature, blood oxygen saturation level, etc.) and may similarly report the data to other devices or systems, such as device 110, server 116, etc. Although not shown, additional sensor devices, such as a network-connected knee brace, elbow brace, and so forth, may similarly be associated with user 190 and may be in communication with device 110 and/or or server 116 via access network(s) 120, telecommunication network 102, and so forth.

As noted above, the access networks 122 may be in communication with a server 116 and a database (DB) 118. The server 116 and DB 118 may be associated with a service, or system for mobility tracking and recovery/therapy progression, as described herein. In accordance with the present disclosure, server 116 may comprise a computing system or server, such as computing system 400 depicted in FIG. 4, and may be configured to provide one or more operations or functions for presenting a notification of an advancement along a therapy progression based on an output of a recovery model in accordance with a plurality of inputs comprising at least a visual input from at least one sensor device, as described herein. It should be noted that as used herein, the terms “configure,” and “reconfigure” may refer to programming or loading a processing system with computer-readable/computer-executable instructions, code, and/or programs, e.g., in a distributed or non-distributed memory, which when executed by a processor, or processors, of the processing system within a same device or within distributed devices, may cause the processing system to perform various functions. Such terms may also encompass providing variables, data values, tables, objects, or other data structures or the like which may cause a processing system executing computer-readable instructions, code, and/or programs to function differently depending upon the values of the variables or other data structures that are provided. As referred to herein a “processing system” may comprise a computing device including one or more processors, or cores (e.g., as illustrated in FIG. 4 and discussed below) or multiple computing devices collectively configured to perform various steps, functions, and/or operations in accordance with the present disclosure.

In one example, DB 118 may comprise a physical storage device integrated with server 116 (e.g., a database server), or attached or coupled to the server 116, to store various types of information in support of systems for presenting a notification of an advancement along a therapy progression based on an output of a recovery model in accordance with a plurality of inputs comprising at least a visual input from at least one sensor device, in accordance with the present disclosure. For example, DB 118 may store visual and/or audio data collected from one or more network-connected sensor devices, such as device 110, e.g., video, light detection and ranging (LiDAR) visual data (e.g., a sequence of LiDAR images), and/or audio pertaining to user 190, biometric data of user 190, e.g., collected from devices 111 and/or 112, and so forth. In accordance with the present disclosure, DB 118 may store timelines of user mobility data, e.g., a timeline comprising visual and/or audiovisual clips of a user engaging in one or more motion activities, and which may include additional user mobility/health information in some examples. In one example, DB 118 may store user profiles, including for each user, such as user 190, one or more active monitoring models (e.g., for motion activities that are actively being monitored for user 190) and/or one or more active recovery models, any medical entities or other authorized entities that may access one or more timelines associated with mobility monitoring and/or recovery of the user 190, and so forth. In one example, DB 118 may store monitoring models and recovery models, which may be specific for each user, such as user 190, and/or which may include general models that are may be deployed for various users, e.g., at the direction of such users and/or their healthcare professionals. In one example, DB 118 may further store user's health records which may be used by health professionals to aid in decision-making.

In one example, the monitoring models and/or recovery/therapy models may include various sub-models and/or sub-components. Thus, for example, DB 118 may store information regarding patterns, e.g., signatures, and/or machine learning models for detecting body parts, for detecting particular motions associated with body parts, for detecting conditions of body parts, etc., other instructions, code, variables, or the like in connection with detecting deviations in performance of a motion activity from a representative example, e.g., as indicated by a motion model, or the like, and so forth.

In an illustrative example, server 116 may obtain a first plurality of inputs from at least one sensor device associated with user 190, e.g., including at least video and/or a sequence of LiDAR images from device 110. Server 116 may then apply the first plurality of inputs to a monitoring model for monitoring a particular type of movement activity of user 190. In one example, the monitoring model may be activated in accordance with a user input electing to activate the monitoring model, e.g., to monitor at least one type of motion activity of user 190. In one example, server 116 may engage in generating the monitoring model, e.g., by baselining of user 190 performing examples of the motion activity to generate/train a motion model, etc. In accordance with the present disclosure, the monitoring model may be configured to detect at least one trigger condition in accordance with the first plurality of inputs. Accordingly, server 116 may obtain an output of the monitoring model in accordance with the first plurality of inputs, where the output indicates the at least one trigger condition (e.g., an anomaly) is detected. For instance, the at least one trigger condition may be an acute health issue, e.g., slip-and-fall, tripping, dropping of object on foot, smashing elbow into fixed or other objects, etc. that may be detected via a detection model operating on at least visual data, e.g., obtained from device 110 or the like.

In one example, the at least one trigger condition may be detected via an explicit input or confirmation from the user 190 or a healthcare entity, such as another user via device 113, e.g., definitively indicating that the user 190 has a fractured tibia, etc. Alternatively, or in addition, the at least one trigger condition may comprise a range of motion of user 190 that has deteriorated beyond a threshold deviation as compared to a movement model and/or a detection of an audible utterance of user 190 indicative of pain via one or more detection models operating on audio data. It should be noted that in accordance with the present disclosure, server 116 may record in DB 118 the visual data (e.g., as video/visual clips of the user 190 engaged in the motion activity (or activities) to be monitored). For instance, the visual clips may be presented in a timeline for user 190, e.g., upon request by the user or an authorized healthcare provider or other entities (e.g., a guardian, a parent, etc.).

In response to the at least one trigger condition, in one example, server 116 may activate a recovery model. The recovery model may include a therapy progression for user 190, e.g., expected ranges of motion, degrees or extents of completion of motion, or the like over a course of time, e.g., 30 percent of maximum overhead extension after one week, 75 percent of maximum overhead extension after one month, full recovery after 3 months, etc. The recovery model may include one or more motion models that may be of the same or similar nature as motion models that may be included in the monitoring model deployed for user 190 (or that may be used in other monitoring models for other users). In one example, server 116 may obtain a second plurality of inputs from the at least one sensor device (e.g., at least device 110, and optionally device 111, device 112, or the like). Accordingly, the second plurality of inputs may comprise at least visual data from device 110, and may further include audio, and/or biometric data.

Server 116 may then apply the second plurality of inputs to the recovery model to obtain an output that indicates an advancement along a therapy progression. For example, server 116 may determine in accordance with the recovery model that the user can extend to only 50 percent of a maximum extension, whereas the therapy progression, e.g., a recovery schedule, may indicate that the user 190 is expected to have achieved 75 percent of maximum extension by such time. Server 116 may then present a notification of the advancement along the therapy progression. For instance, the notification may be presented to the user 190, e.g., via device 110 and/or via another device associated with user 190, such as device 112, may be presented to a healthcare entity and/or other authorized entities, e.g., via device 113, and so forth. In one example, the progression may be recorded in DB 118. Similarly, server 116 may record in DB 118 the visual data (e.g., as video/visual clips of the user 190 engaged in the motion activity (or activities) associated with the recovery model/therapy progression). For instance, the visual clips may be presented in a timeline for user 190, e.g., upon request by the user or an authorized healthcare provider or other entities.

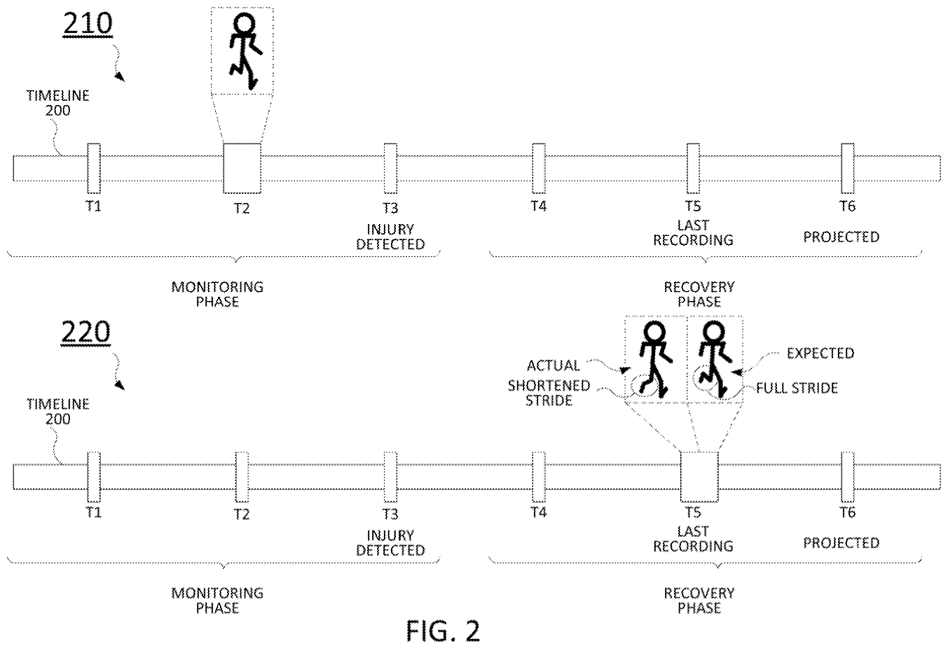

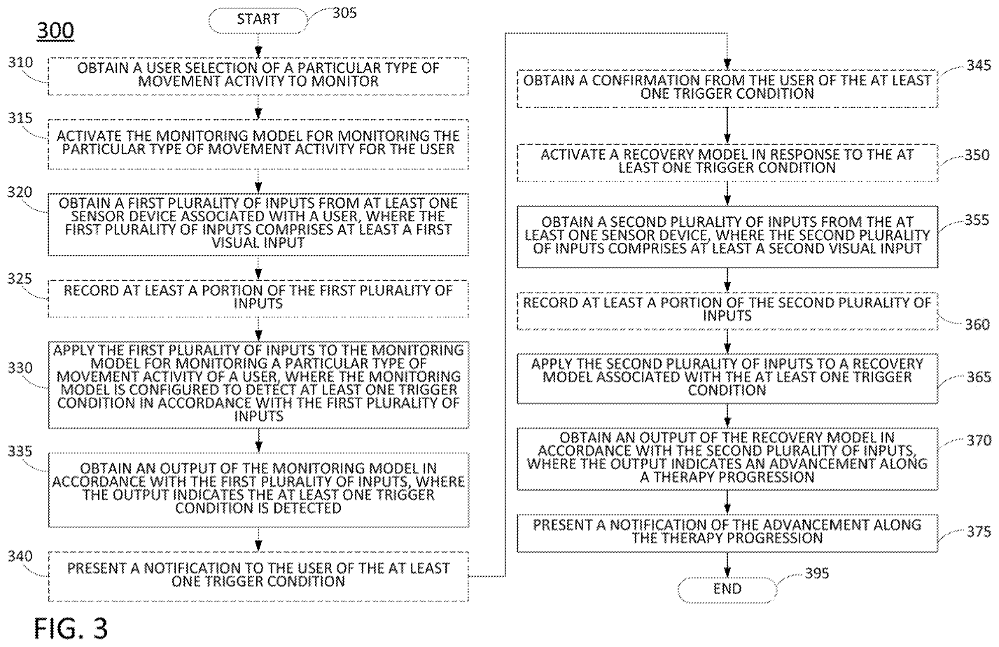

In one example, server 116 may collect and apply visual data (and in one example, additional data such as audio and/or biometric data) to the recovery model on an ongoing basis to continue to track and document the advancement of user 190 along the therapy progression. In one example, the timeline may include visual clips from both before and after a trigger event/condition (e.g., an anomaly). The timeline may be provided to user 190 or other authorized entities upon request, according to a schedule, at other predetermined times, such as when a new visual clip is added to the timeline, when a trigger condition is detected, when a user progresses to a new stage in a therapy progression, and so forth. For instance, an example timeline 200 is illustrated in FIG. 2 and described in greater detail below. In addition, these and other aspects of the present disclosure are discussed in greater detail below in connection with the examples of FIGS. 2 and 3.

In one example, telecommunication network 102 may also include an application server 104 and a database 106. In one example, AS 104 may perform the same or similar functions as server 116. Similarly, DB 106 may store the same or similar information as DB 118, e.g., visual and/or audio data collected from one or more network-connected sensor devices, timelines of user mobility data, user profiles, monitoring models and recovery models, user's health records, and so forth. For instance, telecommunication network 102 may provide a service for mobility tracking and recovery/therapy progression to subscribers, e.g., in addition to television, phone, and/or other telecommunication services. In one example, AS 104, DB 106, server 116, and/or DB 118 may operate in a distributed and/or coordinated manner to perform various steps, functions, and/or operations described herein. In one example, application server 104 may comprise network function virtualization infrastructure (NFVI), e.g., one or more devices or servers that are available as host devices to host virtual machines (VMs), containers, or the like comprising virtual network functions (VNFs). In other words, at least a portion of the network 102 may incorporate software-defined network (SDN) components.

It should be noted that the system 100 has been simplified. Thus, the system 100 may be implemented in a different form than that which is illustrated in FIG. 1, or may be expanded by including additional endpoint devices, access networks, network elements, application servers, etc. without altering the scope of the present disclosure. In addition, system 100 may be altered to omit various elements, substitute elements for devices that perform the same or similar functions, combine elements that are illustrated as separate devices, and/or implement network elements as functions that are spread across several devices that operate collectively as the respective network elements. For example, the system 100 may include other network elements (not shown) such as border elements, routers, switches, policy servers, security devices, gateways, a content distribution network (CDN) and the like. For example, portions of telecommunication network 102 and/or access networks 120 and 122 may comprise a content distribution network (CDN) having ingest servers, edge servers, and the like. In addition, although only a single server 116 and a single DB 118 (and a single AS 104 and DB 106) are illustrated in FIG. 1, it should be noted that any number of servers 116, databases 118, application servers 104, and databases 106 may be deployed. In addition, server 116, DB 118, DB 106, application server 104, and so forth may comprise public or private cloud computing resources, e.g., one or more host devices/servers in one or more data centers to host virtual machines (VMs), containers, or the like comprising various functions, services, and so on. Similarly, although only two access networks 120 and 122 are shown, in other examples, access networks 120 and/or 122 may each comprise a plurality of different access networks that may interface with telecommunication network 102 independently or in a chained manner. For example, device 113 and server 116 may access telecommunication network 102 via different access networks, devices 110-112 may access telecommunication network 102 via different access networks, and so forth. Thus, these and other modifications are all contemplated within the scope of the present disclosure.

FIG. 2 illustrates an example of a timeline 200 of visual data of a user (such as user 190 of FIG. 1) in accordance with the present disclosure. The timeline may be presented to a user engaged in self-monitoring and self-tracking of recovery, and may be presented to a healthcare entity and/or other authorized entities. For instance, the timeline 200 may be presented on a display screen of a user device. In the example of FIG. 2, the timeline 200 may include visual clips over period of time e.g., from time T1 to time T5. The timeline 200 may also indicate one or more future instances of time, such as time T6, where projected visualization of movement may be accessed. The timeline 200 indicates both a monitoring phase (e.g., including times T1-T3) and a recovery phase (e.g., including times T4 and T5, and in the current example, including projected time T6). In one example, and as illustrated in FIG. 2, the timeline 200 may include or may be accompanied by labels for these phases. Each of the indicated times T1-T5 may comprise a time for which a visual clip is available, e.g., a recorded visual clip of the user engaged in at least one motion activity, and may be marked as such, e.g., by a marker that stands-out from a linear bar indicating advancing time along the timeline 200. In this case, the markers may be vertically oriented rectangles, but in other examples could comprise ovals, circles, spheres, colored lines, triangles, etc. In one example, the markers may comprise representative tiles or thumbnail images taken from respective visual clips.

Although the example of FIG. 2 includes a timeline 200 in which the available visual clips are periodic or relatively equally spaced in time, in other, further, and different examples, a timeline may include visual clips that may have a different relationship, or which have no particular time pattern. For instance, the user may not engage in the motion activity according to a fixed schedule, or may not record each instance of the user performing the motion activity. Thus, the available visual clips may appear in the timeline according to the timing of which the visual clips were actually recorded. In the example of FIG. 2, the timeline 200 may include additional labels or markers, such as “injury detected” at time T3. For instance, a marker for a detection of a trigger condition may be included in the timeline 200 at a corresponding time of the detection, and may indicate the type of trigger condition detected, e.g., “injury” and/or a type of injury, deterioration of range of motion beyond threshold, etc. In an example in which the trigger condition is not detected specifically from visual data, such as by a healthcare entity or the user providing a manual input that the user is injured, the label/marker for the detection of the trigger condition may not be aligned with a marker for a visual clip. In the example of FIG. 2, however, the injury detection label is aligned with the marker at time T3, indicating that an injury appears to have been detected via the visual data in the visual clip for time T3.

In a first instance 210 illustrated in FIG. 2, the user or healthcare entity may select to view a clip from time T2. For instance, the user may tap position T2 along the timeline 200 via a touchscreen, may click the position T2 along the timeline 200 using a mouse to navigate an on-screen pointer via a graphical user interface, and so forth. In one example, the selection may cause a larger pop-up of a video/visual clip to appear. In one example, the visual clip may automatically play upon selection. In another example, an additional input, such as tapping within the area of the pop-up may cause the visual clip to begin playing. Thus, for example, the user may wish to see how the user was performing the motion activity prior to injury at time T3 by viewing the visual clip from time T2.

In a second instance 220 illustrated in FIG. 2, the user may select to view a clip from time T5 (e.g., the latest visual clip that is available). In one example, and as illustrated in FIG. 2, the present disclosure may present a recorded visual clip in addition to a visual clip that may present a representative performance of a motion activity according to a therapy progression of a recovery model. For instance, at time T5 the user may be expected to have a full stride of running, e.g., full or nearly full recovery from an injury. Thus, the visual clip of the representative performance may include an expert or the like correctly engaging in the motion activity, e.g., with full stride of running. However, the user at time T5 may not exhibit a full stride, but may continue to engage in a shortened stride, which may be seen in the visual clip recording of the user at time T5.

In one example, the present disclosure may quantify the difference(s) between the user performance of the motion activity at a given instance during the recovery phase and a representative performance of the motion, e.g., a motion model, which may comprise an actual recording of a “correct” performance of the motion activity, or a machine learning model that is generated from a number of representative performances (e.g., positive examples) and in one example additionally in accordance with negative examples (e.g., incorrect performances of the motion activity). For instance, as noted above, a recovery model may comprise one or more motion models for one or more motion activities, as well as a timeline of expectations of the user's performance with respect to the one or more motion activities. A more detailed example of such quantification is described in greater detail below in connection with the example of FIG. 3. In one example, the present disclosure may highlight differences in an actual performance and a representative (e.g., preferred or ideal) performance of a motion activity during the recovery phase. For instance, in the second instance 220, the present disclosure may present side-by-side views of the recorded visual clip and a visual clip of a representative performance, where the difference (e.g., where the user falls short of expectations) is circled, highlighted in a different color, etc. In one example, text explanation may also be provided, such as the explanatory labels of “shortened stride”/“actual” and “full stride”/“expected.”

It should again be noted that FIG. 2 illustrates just one example of a timeline 200 in accordance with the present disclosure, and that in other, further, and different examples, a timeline may have a different form, such as having a different shape, having a different orientation with respect to a display screen, using different markers, and so forth. In addition, a timeline in accordance with the present disclosure may present additional or different information. For instance, in one example, clicking on a marker in the timeline may present, in addition to a visual clip, accompanying audio (e.g., an audiovisual clip). Similarly, clicking on a marker may provide biometric information, such as the user's heart rate, breathing rate, speed, stride length, steps per minute, and so forth, e.g., measured during the time associated with the visual clip. Such information may be presented as averages during the time period, may be presented as instantaneous data that changes with the progression of the visual clip (e.g., frame by frame, or the like), may be presented in chart/graph form, e.g., with a horizontal axis for time extending from the start of the recording of the motion activity to the end and a vertical axis for the corresponding biometric measurements, or for a larger time period of time from which the visual clip was recorded. For instance, the user may run on a treadmill for 30 minutes, but may record 30 seconds for purposes of the monitoring and/or recovery phase tracking. However, biometric data presented in connection with a selection of the visual clip via a timeline may cause biometric data from the entire 30 minutes to be presented. Thus, these and other modifications are all contemplated within the scope of the present disclosure.

FIG. 3 illustrates a flowchart of an example method 300 for presenting a notification of an advancement along a therapy progression based on an output of a recovery model in accordance with a plurality of inputs comprising at least a visual input from at least one sensor device, in accordance with the present disclosure. In one example, the method 300 is performed by a component of the system 100 of FIG. 1, such as by one of the server 116, application server 104, or any of the devices 110 or 111, and/or any one or more components thereof (e.g., a processor, or processors, performing operations stored in and loaded from a memory), or by one or more of the server 116, application server 104, or any one of the devices 110 or 111 in conjunction with one or more other devices, such as a different one or more of server 116, application server 104, or any one of the devices 110-112, and/or one or more of DB 106, DB 118, and so forth. In one example, the steps, functions, or operations of method 300 may be performed by a computing device or system 400, and/or processor 402 as described in connection with FIG. 4 below. For instance, the computing device or system 400 may represent any one or more components of a server 116, application server 104, and/or a device 110 or 111 in FIG. 1 that is/are configured to perform the steps, functions and/or operations of the method 300. Similarly, in one example, the steps, functions, or operations of method 300 may be performed by a processing system comprising one or more computing devices collectively configured to perform various steps, functions, and/or operations of the method 300. For instance, multiple instances of the computing device or system 400 may collectively function as a processing system. For illustrative purposes, the method 300 is described in greater detail below in connection with an example performed by a processing system. The method 300 begins in step 305 and may proceed to optional step 310 or to step 320.

At optional step 310, the processing system may obtain a user selection of a particular type of movement activity to monitor, e.g., from among a plurality of different types of movement activities. The movement activities may include exercise-related movement activities, sports-related movement activities, normal day-to-day movement activities, occupational movement activities, or the like. For instance, exercise-related movement activities may include running, weightlifting (e.g., weighted or unweighted squats, bicep curls, or leg extensions), crunches, etc. Similarly, sports-related movement activities may include a tennis serving motion, for basketball, soccer, or the like shooting, passing, dribbling, etc., and so forth. Day-to-day movement activities may include walking, walking up and down stairs, standing/posture, bending, lifting, picking, etc. Occupational movement activities may include typing, hammering, picking and/or lifting, carrying objects, etc. In one example, the user may select multiple movement activities to monitor. For instance, a user may own a coffee shop where it is typical to bend down and retrieve supplies from an under-counter cabinet. However, it may also be common for the user to lift packages from the ground that are left by a rear entrance. Hence, the user may have two (or more) movement activities to be actively monitored. In one example, the user selection may be obtained via a mobility tracking and recovery/therapy progression application, e.g., on the user's smartphone or the like.

At optional step 315, the processing system may activate a monitoring model for monitoring the particular type of movement activity for the user, e.g., in response to the user selection. In one example, the monitoring model may be selected from among a plurality of available monitoring models, e.g., for different movement activities. It should be noted that insofar as deployment of monitoring models for different movement activities may have associated costs in terms of compute resources, processing time, etc., it may be impractical to continuously monitor and track all possible types of movement activities. As such, the user may specify, which movement activities, if any, should be actively monitored for the user. In one example, step 315 may include generating the monitoring model. For instance, the monitoring model may comprise one or more sub-models, which may include a movement model (e.g., a machine learning model) for detecting a particular type of movement activity. For example, the processing system may record video of the user performing a particular motion once or multiple times. Alternatively, or in addition, the processing system may record LiDAR spatial data (e.g., additional visual data) of the user performing the motion or movement activity. In one example, the motion activity may be a self-directed movement of the user. In one example, the user may designate one or more exemplary movements that the user considers to be “ideal” examples of the movement activity. From these examples, a movement model may be trained. Alternatively, or in addition, in one example, a movement model may be a general model trained by experts, e.g., by medical professionals, such as doctors, physical therapists, etc. for particular types of motion. In one example, the monitoring model may include sub-models for detecting acute health events, e.g., slip-and-fall, tripping, dropping of object on foot, smashing an elbow into fixed or other objects, etc. In one example, the monitoring model may include one or more sub-models for detecting particular utterances that may comprise trigger conditions, e.g., utterances indicative of pain (e.g., “ouch,” “ow,” swear words, etc.), spoken key words or phrases, such as “pain level 3.5,” “record pain level 3.5,” “pain level 3.5 lifting,” “record pain level 3.5 lifting,” etc.

In accordance with the present disclosure, a machine learning model (MLM), e.g., trained in accordance with a machine learning algorithm (MLA), may comprise, for example, a deep learning neural network, or deep neural network (DNN), a generative adversarial network (GAN), a support vector machine (SVM), e.g., a binary, non-binary, or multi-class classifier, a linear or non-linear classifier, and so forth. In one example, the MLA may incorporate an exponential smoothing algorithm (such as double exponential smoothing, triple exponential smoothing, e.g., Holt-Winters smoothing, and so forth), reinforcement learning (e.g., using positive and negative examples after deployment as a MLM), and so forth. It should be noted that various other types of MLAs and/or MLMs may be implemented in examples of the present disclosure, such as k-means clustering and/or k-nearest neighbor (KNN) predictive models, support vector machine (SVM)-based classifiers, e.g., a binary classifier and/or a linear binary classifier, a multi-class classifier, a kernel-based SVM, etc., a distance-based classifier, e.g., a Euclidean distance-based classifier, or the like, and so on. In one example, a trained detection model may be configured to process those features which are determined to be the most distinguishing features of the associated event or object, e.g., those features which are quantitatively the most different from what is considered statistically normal or average from other events or objects that may be detected via a same system, e.g., the top 20 features, the top 50 features, etc.

To illustrate, the processing system may generate (e.g., train) and/or store detection models for detecting types of events (e.g., actions, occurrences, or other scenarios). For instance, the types of events may include “acute” health events, such as “wound,” “slip-and-fall,” “collision,” or the like. The detection models, or signatures, may be specific to particular types of visual/image and/or audio data in the data feed. For instance, with respect to a detection model that uses visual input, the input data may include low-level invariant image data, such as colors (e.g., RGB (red-green-blue) or CYM (cyan-yellow-magenta) raw data (luminance values) from a CCD/photo-sensor array), shapes, color moments, color histograms, edge distribution histograms, etc. Visual features may also relate to movement in a video or other visual sequences (e.g., visual aspects of a data feed of a virtual environment) and may include changes within images and between images in a sequence (e.g., video frames or a sequence of still image shots), such as color histogram differences or a change in color distribution, edge change ratios, standard deviation of pixel intensities, contrast, average brightness, and the like. Alternatively, or in addition, the visual data may also include spatial data, e.g., LiDAR positional data. For instance, a user may be captured in video and/or LiDAR and represented as a point cloud which may comprise a predictor for training one or more machine learning models. In one example, such a point cloud may be reduced, e.g., via feature matching to provide a lesser number of markers/points to speed the processing of training (and classification for a deployed MLM).

As noted above, a monitoring model may include a movement model that is representative of a type of movement activity (e.g., a machine learning-based movement model). Thus, for example, the movement model may comprise a classifier (e.g., a trained MLM) for detecting/classifying whether a movement is/is not of a particular movement type, e.g., a binary classifier to distinguish between walking/not walking, lifting an object/not lifting an object, etc., or may be a multi-class classifier, e.g., a DNN, such as a decision tree, etc. to identify whether a user is “walking,” “running,” “swimming,” “cycling,” “playing tennis,” “picking and placing,” “performing squats,” etc. In one example, the processing system may detect and identify physical markers indicative of limbs and joints of a user. For instance, the user may be represented as a point cloud of major points/markers. The positions of such markers may be recorded in a temporal sequence, e.g., frame by frame, or averaged over several frames (e.g., every 4 frames, every 6 frames, or 10 frames) e.g., depending on the frame rate or other factors, sampled every 5 frames, etc. As such, movement (e.g., from frame to frame or other temporal sequences) may be quantified as a set of displacements of the multitude of points. In one example, the movement model may be used to detect when a user is engaged in a movement activity of a particular type.

In one example, the monitoring model may also include a computer vision (CV) sub-component to detect changes in the movement of the user between instances of the performance of the particular motion activity and/or deviations from the “ideal” performance of the motion activity. For instance, the “ideal” performance of the motion activity may be defined by the movement model. In one example, motion may be estimated using a sequence of co-variance matrices indicative of the difference between point clouds in a temporal sequence, e.g., for a relevant portion of the human body. In one example, this may be averaged over a sliding time window, e.g., to account for the possibility of allowable differences in the timing of performance of a motion activity. However, where speed is relevant to the “correct” performance of the motion activity, the sliding time window may be narrower, for example.

The monitoring model may have one or several trigger conditions that may be monitored for detection, depending on the particular configuration and which model(s) are active. For instance, a trigger condition may comprise a range of motion that has deteriorated beyond a threshold deviation as compared to a movement model, a detection of an acute health event via one or more detection models, and/or a detection of an audible utterance indicative of pain via one or more detection models operating on audio data. In the case of range of motion, differences in point cloud sequences for actual recorded performance and ideal performance (e.g., indicated by the movement model) may be quantified, and may thus indicate differences in the user's performance and the ideal performance of the motion activity, such as not achieving an expected range of motion. Alternatively, or in addition, a deterioration of range of motion may be detected from differences in point cloud sequences for the user's performance from one instance of performing the motion activity to the next (and in one example, over multiple instances).

At step 320, the processing system obtains a first plurality of inputs from at least one sensor device associated with a user, where the first plurality of inputs comprises at least a first visual input. The at least one sensor device may comprise, for example, at least one camera. In one example, the at least one sensor device may alternatively or additionally comprise a LiDAR unit (e.g., a sensor, array, or the like). In one example, the at least one sensor device may further include at least one microphone and/or at least one wearable biometric device, such as a heart rate monitor, an electrocardiogram (EKG) unit, a pedometer, a smart brace (e.g., a knee brace, an elbow brace, or the like). In one example, the user may be directed to place a camera (and/or LiDAR unit) at a distance from where the user will perform a movement activity. In one example, the at least one sensor device may comprise a mobile computing device of the user (e.g., a mobile smartphone, tablet computer, or the like). Alternatively, or in addition, the at least one sensor device may comprise an Internet of Things (IoT) device, such as a network-connected camera, microphone, treadmill, etc. in a fixed or relatively fixed location that is associated with the user, such as in the user's home gym, in the user's workspace, etc. As such, the first plurality of inputs comprises at least the first visual input (e.g., video and/or LiDAR recordings) but may also include audio inputs, and/or biometric data inputs.

At optional step 325, the processing system may record at least a portion of the first plurality of inputs. For instance, as noted above, the user may be recorded performing the motion activity, which may be presented in a timeline format for the user, a healthcare entity, or other authorized entities. In one example, the processing system may comprise a network-based processing system that may receive the first plurality of inputs from a user device.

At step 330, the processing system applies the first plurality of inputs to a monitoring model for monitoring a particular type of movement activity of a user, where the monitoring model is configured to detect at least one trigger condition in accordance with the first plurality of inputs. For instance, the monitoring model may be activated at optional step 315 as described above.

At step 335, the processing system obtains an output of the monitoring model in accordance with the first plurality of inputs, wherein the output indicates the at least one trigger condition (e.g., an anomaly) is detected. As noted above, the at least one trigger condition may comprise a detection of an acute health issue, e.g., slip-and-fall, tripping, dropping of object on foot, smashing elbow into fixed or other object, etc. (broadly an “injury”) that may be detected via a detection model operating on at least visual data. In one example, the at least one trigger condition may comprise a deterioration of a range of motion beyond threshold. For instance, as noted above, the monitoring model may include a movement model that may be used to detect when a user is engaged in a movement activity of a particular type. In addition, the monitoring model may include a computer vision (CV) component to detect changes in the movement of the user between instances of the performance of the particular motion activity and/or deviations from the “ideal” performance of the motion activity. For instance, the “ideal” performance of the motion activity may be defined by the movement model. In addition, the user may be represented as a point cloud of major points/markers. The positions of such markers may be recorded in a temporal sequence, e.g., frame by frame, or averaged over every 4 frames or 10 frames, e.g., depending on the frame rate or other factors, sampled every 5 frames, etc. As such, movement (e.g., from frame to frame or other temporal sequences) may be quantified as a set of displacements of the multitude of points.

In one example, a deviation may be a difference in speed of performance of the motion activity compared to the movement model. In one example, a deviation may be a difference in range of motion from the movement model for one or more limbs of the user either in extension, rotation, or the like. In one example, a deviation that exceeds a threshold may be a trigger condition, e.g., for detecting a health anomaly and for activating a recovery/therapy phase. In one example, a deviation beyond a threshold range over a threshold period of time may be a trigger condition (e.g., user has not passed 70% of full extension over the course of three sessions, over the course of at least 1 week, at least 2 weeks, etc.).

In one example, the deviation, e.g., in speed, distance and/or range of motion in extension, rotation, etc. may be quantified. For instance, a user may be captured in video and/or LiDAR and represented as a point cloud. In one example, the point cloud may be transformed to match an initial pose of the movement model. For instance, the user may start the motion activity turned at a slight angle from the camera as compared to the ideal/head-on view that may associated with the movement model. In one example, a feature extraction may be used to reduce the number of points in the point cloud. In one example, motion may be estimated using a sequence of co-variance matrices indicative of the difference between point clouds or reduced representation in a temporal sequence, e.g., for a relevant portion of the human body. In one example, this may be averaged over a sliding time window, e.g., to account for the possibility of allowable differences in the timing of performance of a motion activity. However, where speed is relevant to the “correct” performance of the motion activity, the sliding time window may be narrower, for example.

Alternatively, or in addition, the monitoring model may include one or more sub-models for detecting particular utterances that may comprise trigger conditions, e.g., utterances indicative of pain (e.g., “ouch,” “ow,” swear words, etc.), spoken key words or phrases, such as “pain level 3.5,” “record pain level 3.5,” “pain level 3.5 lifting,” “record pain level 3.5 lifting,” etc. In view of the foregoing, one of several trigger conditions may be detected via the monitoring model, depending on the particular configuration and which models are active. For instance, a trigger condition may comprise a range of motion that has deteriorated beyond a threshold deviation as compared to a movement model, a detection of an acute health event via one or more detection models, and/or a detection of an audible utterance indicative of pain via one or more detection models operating on audio data.

At optional step 340, the processing system may present a notification to the user of the at least one trigger condition (anomaly). In one example, optional step 340 may comprise presenting a timeline of the at least the portion of the first plurality of inputs. For instance, the timeline may comprise a visual presentation of a linear sequence of selectable audiovisual items, e.g., visual clips (which in one example, may also include audio), or images such as thumbnails or representative images of available video clips. To illustrate, in one example, the processing system may record visual and/or audiovisual data of the user performing the motion activity. In one example, recordings may be collected over time for different instances of the user performing the motion activity. In addition, the recordings, e.g., audiovisual clips, may be presented in a timeline, e.g., a scrollable timeline, from which the user and/or medical entities or other authorized entities, may select clips for presentation. For instance, the timeline may be presented as a sequence of tiles, a line, or a bar (which may show a time scale), where hovering, clicking, etc. over a portion of the timeline via a touchscreen and/or graphical user interface may then show a corresponding audiovisual item corresponding to a time along the line/scale. In one example, the timeline may be the same or similar to at least a portion of the example timeline 200 of FIG. 2 (e.g., at least the monitoring phase).

At optional step 345, the processing system may obtain a confirmation from the user of the at least one trigger condition. For instance, optional steps 340 and 345 may include querying the user, e.g., “impact injury detected—is this correct? Y/N,” and obtaining a response from the user, e.g., via the application associated with the service for mobility tracking and recovery/therapy progression on the user's smartphone or the like. The notification and response/confirmation may be in different formats depending on the trigger condition and/or how the trigger condition was detected. For instance, if a deterioration in a range of motion is detected, the user may be queried: “overhead lift range of motion detected to be limited to 75%. Have you suffered an injury? Y/N.” Depending on the response, the processing system may follow with one or more additional queries, such as: “did you experience pain during this movement? Y/N.” In one example, a visual clip may also be presented for the user to review. In one example, when an injury or another trigger condition is confirmed, optional step 345 may include obtaining a user confirmation to enter a recovery phase (e.g., to activate a recovery model).

At optional step 350, the processing system may activate a recovery model in response to the at least one trigger condition. In one example, the activating may be further in response to a user confirmation obtained at optional step 345. Alternatively, optional step 350 may include obtaining the user consent to activate the recovery model. In one example, optional step 350 may include presenting the user with a suggested recovery model, e.g., a recovery model that the processing system intends to activate, and receiving confirmation from the user. In one example, optional step 345 or optional step 350 may include obtaining a healthcare entity confirmation of the trigger condition and/or of the intended recovery model. In accordance with the present disclosure, the recovery model may include a therapy/recovery progression. In one example, the progression may be defined by a healthcare entity for the user. In another example, the therapy progression may be selected by a user, e.g., from among slow, moderate, fast recovery, or the like or 4 weeks recovery, 6 weeks recovery, etc., or where active 30 minutes per day recovery, 1 hour per day recovery, etc.

In one example, the recovery model may be for the same motion activity as the monitoring model/motion model, or may be for one or more different motion activities (e.g., the user can no longer perform the motion activity that caused an injury or that has deteriorated, or the user can continue to perform the motion activity, but it is detected that the capacity is diminished, such that a different motion activity, or activities, should be commenced for remediation). As such, the user may be tracked for progression according to the recovery model. The recovery model may have multiple motion activities to perform in parallel, or in a sequence (e.g., when the user is detected to successfully complete a motion activity over several days, the user may be directed to progress to a more challenging motion that works the same body part(s)). The recovery model may thus comprise motion models that are the same as, or of a same or similar nature as the motion model(s) that may comprise components of the monitoring model (e.g., one or more machine learning-based movement models).

In one example, the recovery model may include a schedule for the user to progress, e.g., expected ranges of motion for one or more aspects of a motion activity/movement, such as 15 percent bend after 1 week, 30 percent bend after 2 weeks, etc. In one example, the progression of percentage of motion may be based on a comparison of physical markers indicative of limbs and joints of the user and their positions during a sequence of the movement activity to a movement/motion model (e.g., an ideal representation of the motion activity). Thus, it may be determined that 15% of a maximum ideal range may be reached, 30% of the maximum ideal range, etc. In one example, not all aspects of a movement/motion activity may be directly germane to a user's recovery. Accordingly, the recovery model may define certain aspects to be important, and thus benchmarked, and others to be inconsequential. For instance, a motion activity that is part of the recovery model may include a twist and extension. The twist may be incidental, whereas the extension is what needs to be rehabilitated. Thus, the fact that the user may demonstrate full rotation may be inconsequential, whereas the range and/or degree of extension is measured and compared to the ideal standard. In any case, the recovery model may be configured to generate an output indicative of whether the user is on schedule, behind schedule, or ahead of schedule of the therapy progression/sequence for each instance of the user performing the motion activity that is part of the recovery model.

In one example, the recovery model may be selected from a catalog of recovery models with different therapy progressions. In one example, the recovery models may be generated by experts, e.g., healthcare professionals such as physical therapists, or the like. In one example, a user's designated health professional may confirm a selected recovery model for activation and use by the user. In one example, the user and/or the health professional may select from among several options, such as “fast,” “standard,” or “slow” recovery for a particular type of therapy progression. The motion activity or motion activities may be the same, but the expected progression may be several weeks longer or shorter depending on the chosen option. In addition, the expected number of times per day or week of the motion activity, or the expected number of sets and/or repetitions per session may be different depending upon the variation of the recovery model that is selected.

At step 355, the processing system may obtain a second plurality of inputs from the at least one sensor device, where the second plurality of inputs comprises at least a second visual input. For instance, step 355 may comprise similar operations to step 320 described above. However, in one example, during the recovery phase, the user may additionally be instructed or reminded to engage in one or more movement activities in accordance with the recovery model. For instance, the user may be expected to complete 3 sets of 10 repetitions of overhead lifts daily. The user may be queried if the user has performed this task. In one example, each instance of the user performing the motion activity may be expected or suggested to be recorded, e.g., captured via at least one camera and provided to the processing system. In another example, the user may have an expected schedule for performing the motion activity, but may be directed to record the motion activity at a lesser frequency, e.g., at a minimum, every third day, or the like. As such, the user may arrange a mobile computing device or portable camera to provide such recording, or the user may ensure that the motion activity is performed in a location with an accessible fixed camera (and/or other sensor devices), etc.

At optional step 360, the processing system may record at least a portion of the second plurality of inputs. For instance, optional step 360 may comprise similar operations as described above in connection with optional step 325. In this regard, it should again be noted that the user may be recorded performing one or more motion activities, which may be presented in a timeline format for the user, a healthcare entity, or other authorized entities.

At step 365, the processing system applies the second plurality of inputs to the recovery model (e.g., the recovery model being associated with the at least one trigger condition). For instance, as noted above, the recovery model may be activated in response to the at least one trigger condition. As noted above, the recovery model (and hence the processing system implementing the recovery model) may be configured to determine an advancement along a therapy progression in accordance with the second plurality of inputs comprising at least the second visual input. In one example, the second plurality of inputs may include sets of input data obtained at different times. For instance, in one example, the operations of step 365 may be performed on an ongoing basis for instances of the user performing one or more motion activities in accordance with the recovery model.

At step 370, the processing system obtains an output of the recovery model in accordance with the second plurality of inputs, where the output indicates an advancement along the therapy progression. For instance, the “ideal” performance of a motion activity (or a performance of a motion activity to the extent of an expected therapy progression) may be defined by a movement model. In addition, the user may be represented as a point cloud of major points/markers. The positions of such markers may be recorded in a temporal sequence, e.g., frame by frame, or averaged over every 4 frames or 10 frames, e.g., depending on the frame rate or other factors, sampled every 5 frames, etc. As such, movement (e.g., from frame to frame or other temporal sequences) may be quantified as a set of displacements of the multitude of points.

In one example, a deviation may be a difference in speed of performance of the motion activity compared to the movement model. In one example, a deviation may be a difference in range of motion from the movement model for one or more limbs of the user either in extension, rotation, or the like. In one example, a small deviation (e.g., below a threshold) may indicate that the user is performing the motion activity correctly, or as well as to be expected according to a schedule. For example, a user may be expected to achieve 15 percent elbow bend after 1 week, 30 percent bend after 2 weeks, etc. In one example, the progression of percentage of motion may be based on a comparison of physical markers indicative of limbs and joints of the user and their positions during a sequence of the movement activity to a movement/motion model (e.g., an ideal representation of the motion activity). Thus, it may be determined that 15% of a maximum ideal range may be reached, 30% of the maximum ideal range, etc. Accordingly, at step 370, the processing system may identify a difference in the user's performance of a motion activity compared to an expected performance, and may thus identify the user's advancement along the therapy progression, e.g., whether the user has failed, met, or exceed the expectations.

At step 375, the processing system presents a notification of the advancement along the therapy progression. For instance, this may include providing a notification to the user, e.g., via the application associated with the service for mobility tracking and recovery/therapy progression on the user's smartphone or the like, as to whether the user has failed, met, or exceed the expectations at a given instance associated with the second plurality of inputs. In one example, the notification of the advancement along the therapy progression may be presented to a healthcare entity or an authorized non-healthcare entity, such as an insurance entity, a caregiver, etc., e.g., in accordance with a user consent.

In one example, step 375 may comprise presenting a timeline of at least the portion of the second plurality of inputs. For instance, the timeline may comprise a visual presentation of a linear sequence of selectable audiovisual items, e.g., visual clips (which in one example, may also include audio), or images such as thumbnails or representative images of available video clips. For instance, in one example, the processing system may record visual and/or audiovisual data of the user performing the motion activity or motion activities that is/are part of the recovery model (e.g., in accordance with optional step 360). In one example, recordings may be collected over time for different instances of the user performing the motion activity, or activities. In addition, the recordings, e.g., audiovisual clips, may be presented in a timeline, e.g., a scrollable timeline, from which the user and/or medical entities or other authorized entities, may select clips for presentation. Alternatively, or in addition, separate timelines may be presented for different motion activities (e.g., if multiple motion activities are to be performed substantially in parallel, e.g., within a same phase of the therapy progression).

In one example, the timeline may be presented as a sequence of tiles, a line, or a bar (which may show a time scale), where hovering, clicking, etc. over a portion of the timeline via a touchscreen and/or graphical user interface may then show a corresponding audiovisual item corresponding to a time along the line/scale. In one example, the timeline may be the same or similar to at least a portion of the example timeline 200 of FIG. 2. In one example, the timeline may include at least the recovery phase, and in one example, may also include the monitoring phase. In other words, the timeline may include audiovisual items from both before and after the trigger condition (e.g., from both the first plurality of inputs and the second plurality of inputs). In one example, the timeline may include or be accompanied by indicators of expected advancement along therapy progression (e.g., expected at week 1, expected at week 2, etc.). For instance, this may include images/video of ideal motion, images/video of other subjects that are considered exemplary and which may have been selected for presentation via the timeline, or the like.

Following step 375, the method 300 proceeds to step 395 where the method ends.

It should be noted that the method 300 may be expanded to include additional steps, or may be modified to replace steps with different steps, to combine steps, to omit steps, to perform steps in a different order, and so forth. For instance, in one example, the processing system may repeat one or more steps of the method 300, such as steps 320-330 or steps 320-335, e.g., until a trigger condition is detected, steps 310-375 or steps 320-375 for additional monitoring models for different motion activities, etc. In one example, the recovery model (as well as the monitoring model) may include optional inputs relating to a smart brace or the like, e.g., how deep is a knee or elbow bent. In one example, a motion model may be generated to account for this, such as by having an expert perform the motion activity wearing such a brace, in addition to being visually recorded via video and/or LiDAR. In one example, a user may provide audible inputs in the form of words, phrases, and/or commands, or non-speech utterances, e.g., sounds of “ow,” “ouch,” “grunts,” etc. For example, a user may reach a maximum extension and say “that's all,” “I can't go any further,” “pain starts here,” “pain level 5,” etc. Alternatively, or in addition, a user may be prompted to provide verbal input or input via a graphical user interface after the motion activity, such as answering questions: “did you experience pain at any time during the motion activity?” If, yes, when: at start, in middle, at end?” Or, “indicate pain level at maximum extension from 1-5.” Another may query “did you extend until onset of pain or tightness?,” “did you stretch beyond initial comfort zone?,” etc.

These verbal inputs may confirm that a user has reached a limit in a range of motion, for example. In addition, depending on the particular motion activity or type of recovery, pain may be indicative that the user is not ready to progress, or a lack of pain may signal that a user is ready to move to a next motion activity in a sequence of the therapy progression or that the user should be expected to progress further in a range of motion at a next instance of performing the motion activity as part of the therapy progression. In various other examples, the method 300 may further include or may be modified to comprise aspects of any of the above-described examples in connection with FIGS. 1 and 2, or as otherwise described in the present disclosure. Thus, these and other modifications are all contemplated within the scope of the present disclosure.

In addition, although not expressly specified above, one or more steps of the method 300 may include a storing, displaying and/or outputting step as required for a particular application. In other words, any data, records, fields, and/or intermediate results discussed in the method can be stored, displayed and/or outputted to another device as required for a particular application. Furthermore, operations, steps, or blocks in FIG. 3 that recite a determining operation or involve a decision do not necessarily require that both branches of the determining operation be practiced. In other words, one of the branches of the determining operation can be deemed as an optional step. However, the use of the term “optional step” is intended to only reflect different variations of a particular illustrative embodiment and is not intended to indicate that steps not labelled as optional steps to be deemed to be essential steps. Furthermore, operations, steps or blocks of the above described method(s) can be combined, separated, and/or performed in a different order from that described above, without departing from the example embodiments of the present disclosure.

FIG. 4 depicts a high-level block diagram of a computing device or processing system specifically programmed to perform the functions described herein. For example, any one or more components or devices illustrated in FIG. 1 or described in connection with the method 300 may be implemented as the processing system 400. As depicted in FIG. 4, the processing system 400 comprises one or more hardware processor elements 402 (e.g., a microprocessor, a central processing unit (CPU) and the like), a memory 404, (e.g., random access memory (RAM), read only memory (ROM), a disk drive, an optical drive, a magnetic drive, and/or a Universal Serial Bus (USB) drive), a module 405 for presenting a notification of an advancement along a therapy progression based on an output of a recovery model in accordance with a plurality of inputs comprising at least a visual input from at least one sensor device, and various input/output devices 406, e.g., a camera, a video camera, storage devices, including but not limited to, a tape drive, a floppy drive, a hard disk drive or a compact disk drive, a receiver, a transmitter, a speaker, a display, a speech synthesizer, an output port, and a user input device (such as a keyboard, a keypad, a mouse, and the like).

Although only one processor element is shown, it should be noted that the computing device may employ a plurality of processor elements. Furthermore, although only one computing device is shown in the Figure, if the method(s) as discussed above is implemented in a distributed or parallel manner for a particular illustrative example, i.e., the steps of the above method(s) or the entire method(s) are implemented across multiple or parallel computing devices, e.g., a processing system, then the computing device of this Figure is intended to represent each of those multiple computers. Furthermore, one or more hardware processors can be utilized in supporting a virtualized or shared computing environment. The virtualized computing environment may support one or more virtual machines representing computers, servers, or other computing devices. In such virtualized virtual machines, hardware components such as hardware processors and computer-readable storage devices may be virtualized or logically represented. The hardware processor 402 can also be configured or programmed to cause other devices to perform one or more operations as discussed above. In other words, the hardware processor 402 may serve the function of a central controller directing other devices to perform the one or more operations as discussed above.

It should be noted that the present disclosure can be implemented in software and/or in a combination of software and hardware, e.g., using application specific integrated circuits (ASIC), a programmable logic array (PLA), including a field-programmable gate array (FPGA), or a state machine deployed on a hardware device, a computing device, or any other hardware equivalents, e.g., computer readable instructions pertaining to the method(s) discussed above can be used to configure a hardware processor to perform the steps, functions and/or operations of the above disclosed method(s). In one example, instructions and data for the present module or process 405 for presenting a notification of an advancement along a therapy progression based on an output of a recovery model in accordance with a plurality of inputs comprising at least a visual input from at least one sensor device (e.g., a software program comprising computer-executable instructions) can be loaded into memory 404 and executed by hardware processor element 402 to implement the steps, functions or operations as discussed above in connection with the example method 300. Furthermore, when a hardware processor executes instructions to perform “operations,” this could include the hardware processor performing the operations directly and/or facilitating, directing, or cooperating with another hardware device or component (e.g., a co-processor and the like) to perform the operations.

The processor executing the computer readable or software instructions relating to the above described method(s) can be perceived as a programmed processor or a specialized processor. As such, the present module 405 for presenting a notification of an advancement along a therapy progression based on an output of a recovery model in accordance with a plurality of inputs comprising at least a visual input from at least one sensor device (including associated data structures) of the present disclosure can be stored on a tangible or physical (broadly non-transitory) computer-readable storage device or medium, e.g., volatile memory, non-volatile memory, ROM memory, RAM memory, magnetic or optical drive, device or diskette and the like. Furthermore, a “tangible” computer-readable storage device or medium comprises a physical device, a hardware device, or a device that is discernible by the touch. More specifically, the computer-readable storage device may comprise any physical devices that provide the ability to store information such as data and/or instructions to be accessed by a processor or a computing device such as a computer or an application server.

While various embodiments have been described above, it should be understood that they have been presented by way of example only, and not limitation. Thus, the breadth and scope of a preferred embodiment should not be limited by any of the above-described example embodiments, but should be defined only in accordance with the following claims and their equivalents.

What is claimed is:

1. A method comprising:
obtaining, by a processing system including at least one processor, a first plurality of inputs from at least one sensor device associated with a user, wherein the first plurality of inputs comprises at least a first visual input;
applying, by the processing system, the first plurality of inputs to a monitoring model for monitoring a particular type of movement activity of the user, wherein the monitoring model is configured to detect at least one trigger condition in accordance with the first plurality of inputs;
obtaining, by the processing system, an output of the monitoring model in accordance with the first plurality of inputs, wherein the output indicates the at least one trigger condition is detected;
obtaining, by the processing system, a second plurality of inputs from the at least one sensor device, wherein the second plurality of inputs comprises at least a second visual input;
applying, by the processing system, the second plurality of inputs to a recovery model associated with the at least one trigger condition;
obtaining, by the processing system, an output of the recovery model in accordance with the second plurality of inputs, wherein the output indicates an advancement along a therapy progression; and
presenting, by the processing system, a notification of the advancement along the therapy progression.

2. The method of claim 1, further comprising:
activating the recovery model in response to the at least one trigger condition.

3. The method of claim 1, further comprising:
presenting a notification to the user of the at least one trigger condition; and
obtaining a confirmation from the user of the at least one trigger condition.

4. The method of claim 3, further comprising:
recording at least a portion of the first plurality of inputs.

5. The method of claim 4, wherein the presenting of the notification of the at least one trigger condition comprises presenting a timeline of the at least the portion of the first plurality of inputs.

6. The method of claim 5, wherein the timeline comprises a visual presentation of a linear sequence of selectable audiovisual items.

7. The method of claim 1, further comprising:
recording at least a portion of the second plurality of inputs.

8. The method of claim 7, wherein the presenting of the notification of the advancement along the therapy progression comprises presenting a timeline of the at least the portion of the second plurality of inputs.

9. The method of claim 8, further comprising:
recording at least a portion of the first plurality of inputs, wherein the timeline further includes the at least the portion of the first plurality of inputs.

10. The method of claim 8, wherein the timeline comprises a visual presentation of a linear sequence of selectable audiovisual items.

11. The method of claim 1, wherein the notification of the advancement along the therapy progression is presented to at least one of:
the user;
a healthcare entity; or
an authorized non-healthcare entity.

12. The method of claim 1, wherein the monitoring model comprises a machine learning-based movement model.

13. The method of claim 1, wherein the recovery model comprises a machine learning-based movement model.

14. The method of claim 1, wherein the first plurality of inputs or the second plurality of inputs further comprises at least one of:
audio inputs; or
biometric data inputs.

15. The method of claim 1, wherein the at least one sensor device comprises at least one camera, and wherein the at least one sensor device further comprises at least one of: at least one microphone or at least one wearable biometric device.

16. The method of claim 1, wherein the at least one trigger condition comprises:
an injury; or
a deterioration of a range of motion beyond a threshold.

17. The method of claim 1, further comprising:
obtaining a user selection of the particular type of movement activity to monitor; and
activating the monitoring model for monitoring the particular type of movement activity for the user.

18. The method of claim 1, wherein the particular type of movement activity comprises one of:
an exercise activity type;
a day-to-day movement activity type; or
an occupational movement activity type.

19. A non-transitory computer-readable medium storing instructions which, when executed by a processing system including at least one processor, cause the processing system to perform operations, the operations comprising:
obtaining a first plurality of inputs from at least one sensor device associated with a user, wherein the first plurality of inputs comprises at least a first visual input;
applying the first plurality of inputs to a monitoring model for monitoring a particular type of movement activity of the user, wherein the monitoring model is configured to detect at least one trigger condition in accordance with the first plurality of inputs;
obtaining an output of the monitoring model in accordance with the first plurality of inputs, wherein the output indicates the at least one trigger condition is detected;
obtaining a second plurality of inputs from the at least one sensor device, wherein the second plurality of inputs comprises at least a second visual input;
applying the second plurality of inputs to a recovery model associated with the at least one trigger condition;
obtaining an output of the recovery model in accordance with the second plurality of inputs, wherein the output indicates an advancement along a therapy progression; and
presenting a notification of the advancement along the therapy progression.

20. An apparatus comprising:
a processing system including at least one processor; and
a computer-readable medium storing instructions which, when executed by the processing system, cause the processing system to perform operations, the operations comprising:
obtaining a first plurality of inputs from at least one sensor device associated with a user, wherein the first plurality of inputs comprises at least a first visual input;
applying the first plurality of inputs to a monitoring model for monitoring a particular type of movement activity of the user, wherein the monitoring model is configured to detect at least one trigger condition in accordance with the first plurality of inputs;
obtaining an output of the monitoring model in accordance with the first plurality of inputs, wherein the output indicates the at least one trigger condition is detected;
obtaining a second plurality of inputs from the at least one sensor device, wherein the second plurality of inputs comprises at least a second visual input;
applying the second plurality of inputs to a recovery model associated with the at least one trigger condition;
obtaining an output of the recovery model in accordance with the second plurality of inputs, wherein the output indicates an advancement along a therapy progression; and
presenting a notification of the advancement along the therapy progression.
